(19) United States
(12) Patent Application Publication
(10) Pub. No.: US 2007/0038040 A1
(43) Pub. Date: Feb. 15, 2007

(54) Arrangements, systems and methods capable of providing spectral-domain polarization-sensitive optical coherence tomography

(75) Inventors: Abraham Cense, Bloomington, IN (US); Mircea Mujat, Medford, MA (US); Boris Hyle Park, Somerville, MA (US); Johannes F. Deboer, Somerville, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation

(21) Appl. No.: 11/410,937

(22) Filed: Apr. 24, 2006

Related U.S. Application Data
(60) Provisional application No. 60/674,008, filed on Apr. 22, 2005.

(51) Int. Cl. A61B 5/00

(52) U.S. Cl. 600/310

(57) ABSTRACT

Systems, arrangements and methods for separating an electromagnetic radiation and obtaining information for a sample using an electromagnetic radiation are provided. In particular, the electromagnetic radiation can be separated into at least one first portion and at least one second portion according to at least one polarization and at least one wave-length of the electromagnetic radiation. The first and second separated portions may be simultaneously detected. Further, a first radiation can be obtained from the sample and a second radiation may be obtained from a reference, and the first and second radiations may be combined to form a further radiation, with the first and second radiations being associated with the electro-magnetic radiation. The information is provided as a function of first and second portions of the further radiations that have been previously separated and can be analyzed to extract birefringent information characterizing the sample.

### CROSS-REFERENCE TO RELATED APPLICATION(S)

This application is based upon and claims the benefit of priority from U.S. Patent Application Ser. No. 60/674,008, filed Apr. 22, 2005, the entire disclosure of which is incorporated herein by reference.

### STATEMENT REGARDING FEDERALLY SPONSORED RESEARCH

The invention was made with the U.S. Government support under Contract No. RO1 EY014975 and RO1RR019768 awarded by the National Institute of Health, and Contract No. F49620-021-1-0014 awarded by the Department of Defense. Thus, the U.S. Government has certain rights in the invention.

### FIELD OF THE INVENTION

The present invention relates to optical imaging, and more particularly to arrangements, systems and methods which are capable of providing spectral-domain polarization-sensitive optical coherence tomography. BACKGROUND OF THE INVENTION

The acquisition speed of a polarization-sensitive optical coherence tomography (PS-OCT) system can be significantly increased by replacing time-domain technology, examples of which are described in J. F. de Boer et al., “Two-dimensional birefringence imaging in biological tissue by polarization-sensitive optical coherence tomography,” Optics Letters, 1997, Vol. 22(12): pp. 934-936, and B. H. Park et al., “Real-time multi-functional optical coherence tomography,” Optics Express, 2003, Vol. 11(7): pp. 782-793.

One exemplary spectral-domain (SD) fiber-based system has been described in N. Nassif et al., “In vivo human retinal imaging by ultrahigh-speed spectral domain optical coherence tomography,” Optics Letters, 2004. Vol. 29(5): pp. 480-482, and N. A. Nassif et al., “In vivo high-resolution video-rate spectral-domain optical coherence tomography of the human retina and optic nerve,” Optics Express, 2004. Vol. 12(3): pp. 367-376. These publications describe the advantages of spectral-domain over time-domain analysis, such as, e.g., faster data acquisition and improved signal-to-noise ratio. For example, the structural information, i.e., the depth profile, can be obtained by Fourier transforming the optical spectrum of the interference at the output of a Michelson interferometer.

An exemplary polarization-sensitive time-domain system, as well as a fiber-based system, has also been described in J. F. de Boer et al., “Two-dimensional birefringence imaging in biological tissue by polarization-sensitive optical coherence tomography,” Optics Letters, 1997, Vol. 22(12): pp. 934-936.

For example, it is possible to compare the image quality and polarization-sensitive results obtained with a known time-domain OCT system from healthy volunteers with those of glaucoma patients as described in B. Cense et al., “Thickness and birefringence of retinal nerve fiber layer of healthy and glaucomatous subjects measured with polarization sensitive optical coherence tomography,” Ophthalmic Technologies XIV, 2004. Proceedings of SPIE Vol. 5314: pp. 179-187.

Lower signal-to-noise ratio in images obtained from glaucoma patients was identified as the possible cause of unreliable results. Furthermore, from the analyzed RNFL thickness and double-pass phase retardation per unit depth (DPPR/UD) data obtained from a healthy subject, it was ascertained that a retinal nerve fiber layer (RNFL) thickness of more than 75 μm should be used for a reliable birefringence measurement as described in this publication. Since, as indicated in this publication, most of the measured glaucomatous nerve fiber layer thickness was less than this limit, complete glaucomatous data set could not be retrieved. In addition, the long acquisition time of 6 seconds per scan and 72 seconds for a complete data set with a time-domain system as described in this publication resulted in unreliable data due to involuntary eye motion and data loss caused by frequent blinking.

Birefringence measurements on human skin in vitro and porcine esophagus in vitro using a spectrometer-based Fourier-domain system have been described in Y. Yasuno et al., “Birefiingence imaging of human skin by polarization-sensitive spectral interferometric optical coherence tomography,” Optics Letters, 2002, Vol. 27(20): pp. 1803-1805; and Y. Yasuno et al., “Polarization-sensitive complex Fourier domain optical coherence tomography for Jones matrix imaging of biological samples,” Applied Physics Letters, 2004, Vol. 85(15): pp. 3023-3025. In the publications, the A-line rate of the measurements was not discussed. Measurements have been described on rabbit tendon in vitro using a polarization-sensitive optical frequency-domain imaging (OFDI) system, provided in J. Zhang et al., “Full range polarization-sensitive Fourier domain optical coherence tomography,” Optics Express, 2004, Vol. 12(24): pp. 6033-6039. The A-line rate of such system was 250 Hz, which was likely not an improvement compared to classic time-domain PS-OCT systems. Certain advantages of spectral-domain OCT over time-domain OCT, which are a higher sensitivity and higher acquisition rate, were not demonstrated by the above-described publications. These improvements are preferable for in-vivo measurements. Described herein below are certain advantages which can be obtained by measuring the thickness and DPPR/UD of the retinal nerve fiber layer of a glaucoma patient in-vivo.

### OBJECTS AND SUMMARY OF THE INVENTION

One of the objects of the present invention is to overcome certain deficiencies and shortcomings of the prior art systems (including those described herein above), and provide an exemplary embodiment of arrangement, system and method which are capable of providing spectral-domain polarization-sensitive optical coherence tomography. This can be done my implementing spectral-domain (SD) analysis, arrangements, systems and methods in PS-OCT (e.g., PS-SD-OCT arrangements, systems and methods).

For example, polarization-sensitive characteristics of the tissue (such as the sample or the target) being investigated can be obtained by analyzing the interferometric signal from an OCT system simultaneously in two orthogonal polarization channels for two sequentially generated input states of polarization. According to one exemplary embodiment of the present invention, different configurations of the high-speed spectrometer can be used in the exemplary PS-SD-OCT arrangements, systems and methods.

The exemplary embodiment of the PS-SD-OCT system, arrangement and method according to the present invention can combine an ultra-high-speed acquisition and a high sensitivity with the polarization sensitivity. This exemplary combination can improve the reliability of measurements obtained from glaucoma patients.

Therefore, exemplary embodiments of systems, arrangements and methods for separating an electro-magnetic radiation and obtaining information for a sample using an electro-magnetic radiation are provided. In particular, the electromagnetic radiation can be separated into at least one first portion and at least one second portion according to at least one polarization and at least one wave-length of the electromagnetic radiation. The first and second separated portions may be simultaneously detected. Further, a first radiation can be obtained from the sample and a second radiation may be obtained from a reference, and the first and second radiations may be combined to form a further radiation, with the first and second radiations being associated with the electromagnetic radiation. The information as a function of first and second portions of the further radiations that have been previously separated.

According to another exemplary embodiment of the present invention, the detection can be performed using a detection arrangement which can include a single row of detection elements. In addition or alternatively, two detection arrangements can be used, with each of the detection arrangements including a single row of detection elements. Further, the separation can be performed using a first element which is configured to separate the electro-magnetic radiation into the first and second portions based on the polarization, and a second element which is configured to separate the electromagnetic radiation into the first and second portions based on the wave-length. The first element can follow the second element in an optical path of the electromagnetic radiation.

A third light directing element can be provided in the optical path in a proximity of the first and second elements, e.g., between the first and second elements, and/or following the first and second elements in the optical path. In addition or alternatively, further light directing elements can be provided in the optical path following the first and second elements. Each of these further elements can direct at least one of the respective separated portions toward the second element. The second element can follow the first element in an optical path of the electromagnetic radiation. Another arrangement can be provided to control a polarization of the generated electromagnetic radiation.

These and other objects, features and advantages of the present invention will become apparent upon reading the following detailed description of embodiments of the invention, when taken in conjunction with the appended claims.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is a diagram of an exemplary embodiment of a polarization-sensitive spectrometer arrangement with two line-scan cameras in accordance with the present invention;

FIG. 2 is a diagram of an exemplary embodiment of a first configuration of Polarization-sensitive spectrometer with a Wollaston prism in accordance with the present invention;

FIG. 3A is a diagram of an exemplary embodiment of a second configurations of a polarization-sensitive detector in accordance with the present invention that includes a Wollaston, with two orthogonal states being separated after a collimator;

FIG. 3B is a diagram of an exemplary embodiment of a second configurations of a polarization-sensitive detector in accordance with the present invention that includes the Wollaston, with two orthogonal states being separated after a transmission grating;

FIG. 4 is a diagram of another exemplary embodiment of the polarization-sensitive spectrometer with parabolic mirrors in accordance with the present invention;

FIG. 5A is a flow diagram of one exemplary embodiment of a method according to the present invention;

FIG. 5B is graphs showing exemplary synchronized trigger waveforms for the line scan cameras (e.g., line trigger, frame trigger) and driving waveforms for the polarization modulator and fast galvanometer in accordance with the present invention;

FIG. 6 is a block diagram of an exemplary embodiment of a system capable of performing polarization-sensitive spectral-domain optical coherence tomography in accordance with the present invention;

FIG. 7A is an illustration of an exemplary spectrometer configuration for one polarization channel in accordance with the present invention;

FIG. 7B is a flow diagram of another exemplary embodiment of a method according to the present invention;

FIG. 8 is an exemplary pseudo fundus image of an exemplary optic nerve head, reconstructed from a three-dimensional volume set generated using the arrangement, system and/or method in accordance with an exemplary embodiment of the present invention;

FIG. 9 is an exemplary structural intensity image of a circular scan around the optic nerve head of a healthy patient generated using the arrangement, system and/or method in accordance with an exemplary embodiment of the present invention;

FIG. 10A is a first exemplary graph illustrating a thickness and double-pass phase retardation (DPPR) of sectors temporal to ONH generated using the arrangement, system and/or method in accordance with an exemplary embodiment of the present invention;

FIG. 10B is a first exemplary graph of the thickness and DPPR of sectors superior to the ONH generated using the arrangement, system and/or method in accordance with an exemplary embodiment of the present invention;

FIGS. 11A-11F are exemplary graphs of RNFL thickness and DPPR per unit density (UD) measurements obtained at different integration times generated using the arrangement, system and/or method in accordance with an exemplary embodiment of the present invention;

FIG. 12 is an exemplary structural intensity image from a circular scan around the optic nerve head of a particular glaucoma patient generated using the arrangement, system and/or method in accordance with an exemplary embodiment of the present invention;

FIG. 13A is a second exemplary graph of the thickness and DPPR of sectors temporal to the ONH generated using the arrangement, system and/or method in accordance with an exemplary embodiment of the present invention;

FIG. 13B is a second exemplary graph of the thickness and DPPR of the sectors superior to the ONH generated using the arrangement, system and/or method in accordance with an exemplary embodiment of the present invention;

FIG. 14 is a further exemplary graph showing the thickness and DPPR in a sector that is part of a field defect in the inferior area of the glaucoma patient generated using the arrangement, system and/or method in accordance with an exemplary embodiment of the present invention;

FIG. 15A is an exemplary graph showing a retinal nerve fiber layer (RNFL) thickness from a nerve fiber layer tissue of the glaucoma patient generated using the arrangement, system and/or method in accordance with an exemplary embodiment of the present invention;

FIG. 15B is an exemplary graph showing DPPR/UD values from nerve fiber layer tissue of the glaucoma patient;

FIG. 15B is an exemplary graph showing DPPR/UD values from nerve fiber layer tissue of the glaucoma patient;

FIG. 16A is an exemplary graph showing Thickness (dotted line) and DPPR (solid line) plots of an area nasal to the ONH of the glaucoma patient;

FIG. 16B is an exemplary graph showing Thickness (dotted line) and DPPR (solid line) plots of an area superior to the ONH of the glaucoma patient;

FIG. 16C is an exemplary graph showing Thickness (dotted line) and DPPR (solid line) plots of an area inferior to the ONH of the glaucoma patient;

FIG. 17A is an exemplary graph showing the RNFL thickness from the nerve fiber layer tissue of the glaucoma patient; and

FIG. 17B is an exemplary graph showing the DPPR/UD values from the nerve fiber layer tissue of the glaucoma patient.

Throughout the figures, the same reference numerals and characters, unless otherwise stated, are used to denote like features, elements, components or portions of the illustrated embodiments. Moreover, while the present invention will now be described in detail with reference to the figures, it is done so in connection with the illustrative embodiments.

### DETAILED DESCRIPTION

### First Exemplary Configuration of Exemplary Embodiments

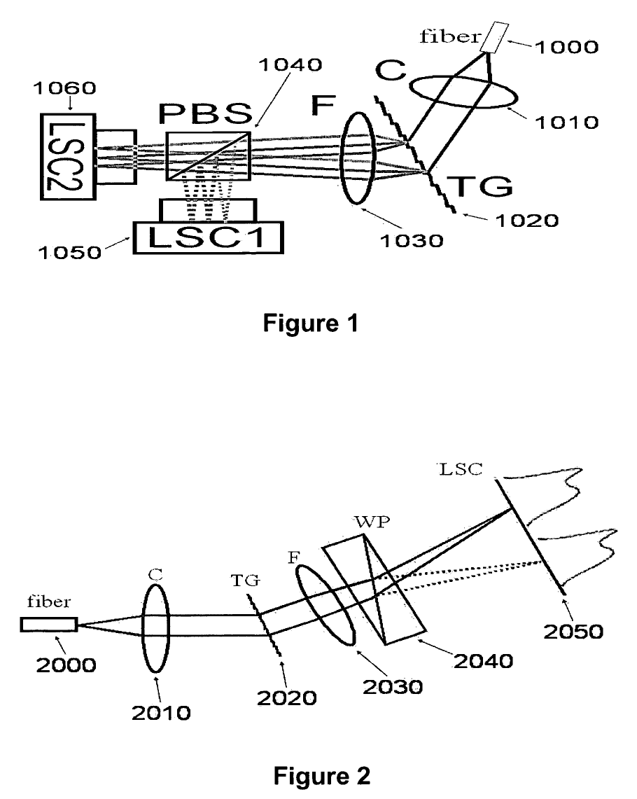

A spectral-domain fiber-based system has been described in N. Nassif et al., “In vivo human retinal imaging by ultrahigh-speed spectral domain optical coherence tomography,” Optics Letters, 2004, Vol. 29(5), pp. 480-482, and N. A. Nassif et al., “In vivo high-resolution video-rate spectral-domain optical coherence tomography of the human retina and optic nerve,” Optics Express, 2004, Vol. 12(3), pp. 367-376. An exemplary embodiment of a system and an arrangement according to the present invention is shown in FIG. 1. As shown therein, the system may include a polarization-sensitive spectrometer with two line-scan cameras. The exemplary system/arrangement can include line-scan cameras 1 and 2 (LSC1:1050, LSC2:1060, respectively), a polarizing beam splitter (PBS:1040), a focuser (F:1030), a transmission grating (TG:1020) and a collimator (C:1010). A clean-up polarizer can be provided in front of a line-scan camera 1 (LSC1:1050), but is not shown in the figure. The exemplary system arrangement according to the present invention modifies a previous system described above to be polarization-sensitive with the use of the line-scan camera 1 (LSC1:1050), the polarizing beam splitter (1040) and a clean-up polarizer in the detector arm, and a polarization modulator in the source arm.

For example, the two orthogonal components of the state of polarization of light or electromagnetic radiation at the end of the fiber in the detection arm can be separated with the polarizing beam splitter (PBS:1040), after which each polarization component can be imaged on its own optical component and/or camera. Since the polarizing beam splitter (PBS:1040) performance is not ideal, some of the polarized light/electro-magnetic radiation that is forwarded to an off-axis camera (which may be included in the polarization beam splitter PBS:1040) may be contaminated with the light/electro-magnetic radiation that has the other polarization state. It therefore should be improved (or cleaned) using an extra polarizer.

### Second Exemplary Configuration of Exemplary Embodiments

Another exemplary embodiment of the system/arrangement according to the present invention is shown in FIG. 2, which illustrates an exemplary polarization-sensitive spectrometer with a Wollaston prism. In this exemplary embodiment, two polarization components are separated by a Wollaston prism (WP:2040), and imaged on a line scan camera (LSC:2050). This exemplary system/arrangement includes a transmission grating (TG:2020), a collimator (C:2010), and a focusing arrangement (F:2030). In use, the camera can record, e.g., two spectra as shown in FIG. 2. Indeed, the prior art systems/arrangements can be made polarization-sensitive by using a Wollaston prism (2040) or a Rochon, Glan-Thomson polarizing element, with a single camera (2050) or multiple cameras. These polarizing elements can spatially separate the two orthogonal polarization components.

For example, by selecting the splitting angle of a Wollaston prism (WP:2040), the two spectra can be spatially separated such that they can both be imaged on the same line-scan camera (2050) simultaneously. This exemplary configuration can use a single camera (2050), thereby simplifying the design of the system/arrangement and possibly reducing costs. Another possible advantage of this exemplary embodiment is that the Wollaston prism (2040) can separate the orthogonal polarization components with a significantly higher extinction ratio than performed by conventional polarizing beam splitters. Therefore, a clean-up polarizer does not have to be implemented, thus further possibly reducing costs, as well as improving the efficiency of the spectrometer by reducing optical losses.

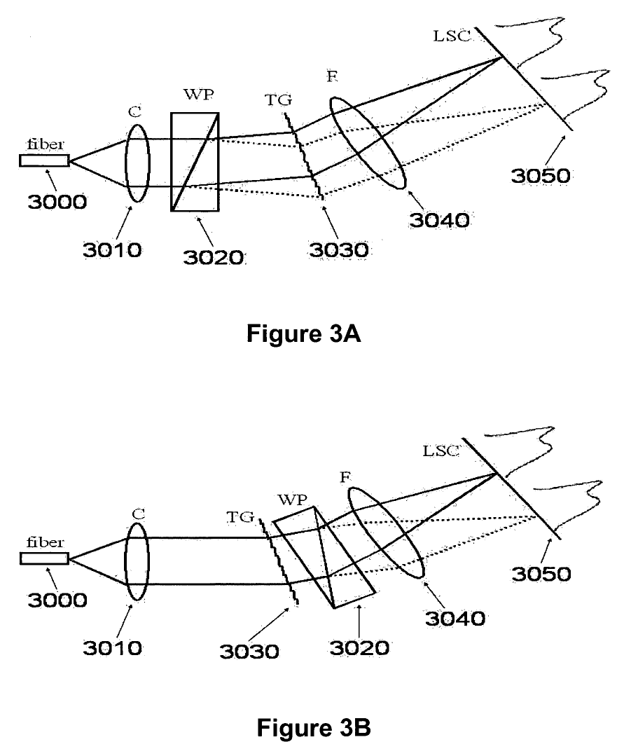

Other exemplary embodiments of the present invention are shown in FIGS. 3A and 3B, which illustrate further exemplary polarization-sensitive spectrometers with a Wollaston prisms. In FIG. 3A, two orthogonal states are separated directly after a collimator (3010). In the FIG. 3B, these two states are separated after a transmission grating (TG:3030). For example, the Wollaston prism (3020) can also be positioned directly after the collimator (3010) or between a diffraction grating (3030) and a focusing arrangement (3040). Depending on the selection of the location, the separation angle of the Wollaston prism (3020) can to be chosen accordingly.

### Third Exemplary Configuration of Exemplary Embodiments

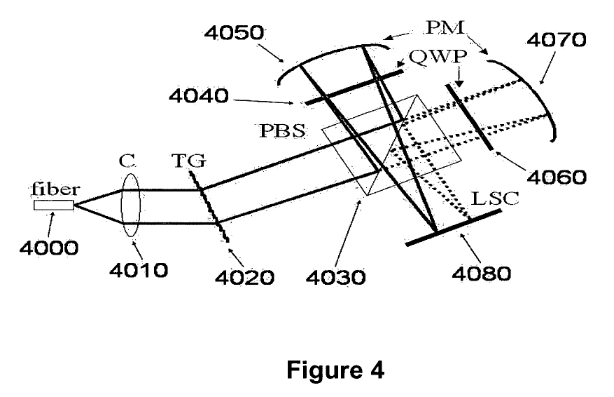

A further exemplary embodiment of the system/arrangement according to the present invention is shown in FIG. 4, which illustrates that parabolic mirrors (4050, 4070) can be used instead of a focusing lens described above in FIGS. 2, 3A and 3B to image the spectra of the two orthogonal polarization components, which can also utilize a single camera (4080) as shown in FIG. 4 or multiple cameras. One of the advantages of this exemplary embodiment is that chromatic aberrations may be reduced, since parabolic mirrors generally do not induce a chromatic dispersion. Other types of aberration such as spherical aberration are likely also minimized.

Similarly to the first and second exemplary configurations described above, the collimator C (4010) can collimate the light/electro-magnetic radiation emerging from the fiber (4000). The light/electro-magnetic radiation can then be dispersed using a transmission grating (4020), and the two orthogonal polarization components may be separated using a polarizing beam splitter (PBS:4030). The two linear polarization components can be transformed into circular polarizations by two achromatic quarter-wave plates (QWP: 4040, 4060). After these two linear polarization components are reflected by the parabolic mirrors (4050, 4070), they are transformed back into linear polarizations using the same achromatic quarter-wave plates (QWP: 4040, 4060). These linear polarizations generally become orthogonal to the initial components, and can therefore be processed differently by the PBS (4030). The linear polarization that has been initially reflected by the PBS (4030) can then be transmitted toward the LSC (4080), while the linear component which has been initially transmitted by the PBS (4030) can be reflected toward the same LSC (4080). The spectra of the two polarization components may be separated using the LSC (4080) by slightly tilting the two mirrors.

Another advantage of this exemplary configuration may be that the light/electro-magnetic radiation generally travels twice through the PBS (4030), and therefore, the polarization purity can be significantly improved without using an additional clean-up polarizer.

The spectra of the two orthogonal polarization components can be imaged on the same LSC in the second and third configurations. If another exemplary arrangement is used, the two spectra can be imaged along parallel lines of a rectangular CCD. Such exemplary arrangement may be advantageous in that off-axis geometrical aberrations may likely be reduced.

In the above-described exemplary configurations, the two acquired spectra can be stored to a hard disk (or another storage device), and analyzed in real time and during post-processing.

For the analysis of these spectra, it is preferable to avoid “ghost birefringence” artifacts. Ghost birefringence is birefringence that is measured by the system, but likely does not exist in reality. It can be caused by an incorrect calibration of the polarization-sensitive spectrometer. The exemplary embodiments of the system, arrangement and method of the present invention provides a procedure for providing a correct calibration of the spectrometer, as described in further detail below.

### Further Configuration(s) and Techniques of Exemplary Embodiments

As described above with reference to the first, second and third exemplary configuration in accordance with the exemplary embodiments of the present invention, a conventional spectral-domain optical coherence tomography system can be made polarization-sensitive. For example, this can be done by adding a polarization modulator in the source arm and a polarizing beam splitter (CVI) combined with a further line scan camera (e.g., Basler, 2048 elements of 10 by 10 μm, maximum line frequency 29,300 Hz) in the detection arm. A high-power superluminescent diode (e.g., SLD-371-HP, Superlum, λ₀=840 nm, Δλ_{FWHM}=50 nm) can be isolated using a broadband isolator (OFR). At the output of the isolator, the light/electro-magnetic radiation can likely be linearly polarized.

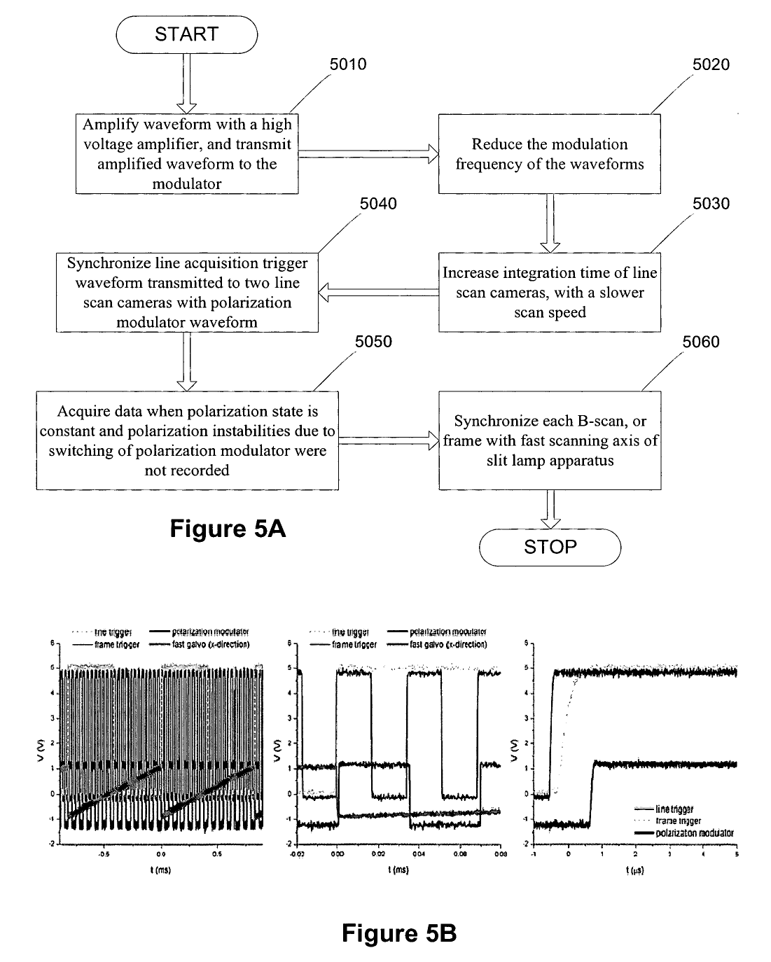

A processing arrangement according to yet another exemplary embodiment of the present invention can be used to generate driving waveforms for line acquisition triggering and for the polarization modulator, which may be positioned either directly or indirectly following the isolator. One exemplary embodiment of the method according to the present invention is shown in FIG. 5A. In particular, the waveform can be amplified with a high voltage amplifier, and may be transmitted to the modulator (step 5010). The waveform can include consist a block wave with, e.g., a maximum frequency of 29,300 Hz, such that two different polarization states perpendicular in a Poincaré sphere representation are produced. The modulation frequency of the waveforms can be slowed down arbitrarily (step 5020), to increase the measurement sensitivity as desired. The integration time of the line scan cameras may be increased accordingly, with a slower scan speed (step 5030). The line acquisition trigger waveform transmitted to the two line scan cameras may be synchronized with the polarization modulator waveform such that consecutive depth scans (A-lines) were acquired with alternating input polarization states (step 5040). Data was only acquired when the polarization state was constant and polarization instabilities due to switching of the polarization modulator were not recorded by shortening the acquisition time of the two cameras to 33 μs (step 5050). Each B-scan, or frame, was synchronized with the fast scanning axis of the slit lamp apparatus(step 5060). This exemplary procedure can be used with the technique and system described in B. Cense et al., “In vivo birefringence and thickness measurements of the human retinal nerve fiber layer using polarization-sensitive optical coherence tomography,” Journal of Biomedical Optics, 2004, Vol. 9(1), pp. 121-125.

FIG. 5B shows graphs showing exemplary synchronized trigger waveforms for the line scan cameras (e.g., line trigger, frame trigger) and driving waveforms for the polarization modulator and fast galvanometer in accordance with the present invention. From left to right, graphs shown in FIG. 5B are provided at a shortened time scale. The trigger and driving waveforms illustrated in FIG. 5B are provided for an exemplary configuration where 20 A-lines were acquired for one image. Within this frame, 20 pulses can be generated to trigger both line scan cameras for the acquisition of 20 spectra. This also can occur at every up flank. Since an internal delay in the camera may be 2 μs, and a 1 μs delay in the polarization modulator, the polarization modulator signal can be delayed in software by approximately 1 μs. It should be understood that 1000 spectra or more can be recorded per cycle of the fast galvanometer. A time delay between the starting points of the different waveforms (right plot) can be generated to compensate for delays in the line scan cameras and the polarization modulator.

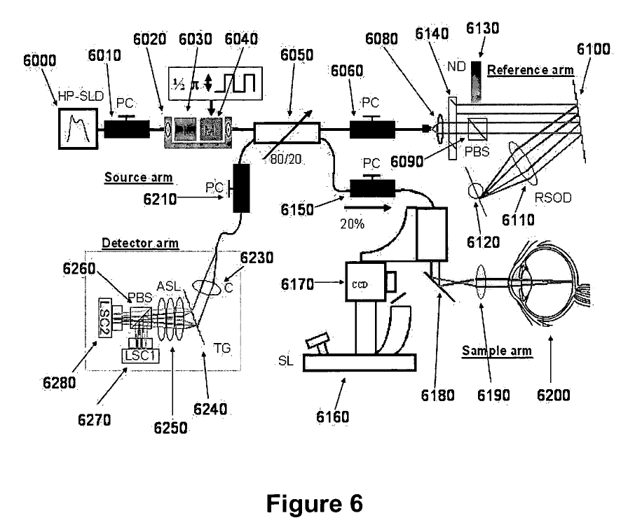

Another exemplary embodiment of the system that is capable of performing polarization-sensitive spectral-domain optical coherence tomography in accordance with the present invention is shown in FIG. 6. In particular, light (or electromagnetic radiation) provided from a broadband source (HP-SLD:6000) can be coupled through an isolator (I:6030) and modulated at 29,300 Hz with a bulk polarization modulator (M:6040). The isolator (I:6030) and the polarization modulator (M:6040) may be placed on a fiber bench (6020). An 80/20 fiber coupler (6050) can distribute the modulated light over the sample and reference arms. The retina may be scanned with a slit lamp (SL:6160) based retinal scanner, and the reference arm can include a rapid scanning delay line (RSOD:6080-6140), that may be used with a polarizing beam splitter (PBS:6090) to ensure equal transmission for both polarization states. A variable neutral density filter (ND:6130) may also be provided for an attenuation. On the return path, interference fringes may be detected using a high-speed polarization-sensitive spectrometer (elements 6230-6280). The light can be collimated (e.g., using element C:6230, −f=60 mm), and diffracted with a transmission grating (TG:6240, 1200 lines/mm) after which a lens (ASL:6250−f=100 mm) can focus the spectra on two line scan cameras (LSC1:6270 and 2:6280). A polarizing beam splitter (6260) in the detection path directed orthogonal polarization components to two cameras (6270, 6280), which may be synchronized with each other and with the polarization modulator (6040) in the source arm. A clean-up polarizer can be positioned in front of LSCl (6270) to remove the contaminating polarization state. Polarization controllers (PC:6010, 6060, 6150, 6210) can be used to fine-tune the polarization state of the light.

For example, the 80/20 fiber coupler (6050) can provided 80% of the power to the reference arm. The rapid scanning delay line (RSOD:6080-6140) can be used with the polarizing beam splitter (6090), to facilitate a transmission of, e.g., equal amounts of power through the delay line for both input polarization states. The RSOD can be used for dispersion compensation, and the galvanometer mirror (6120) may be kept stationary for these measurements. The light returning from the RSOD can be interfered with the light returning from the sample arm. The interference spectra may be recorded with the polarization-sensitive spectrometer in the detection arm, where the two line scan cameras (6270, 6280) may be positioned around the polarizing beam splitter (6260). The light emerging from the fiber may be first collimated (6230) and diffracted with the transmission grating (6240), after which the light can be focused using the lens (6250). The polarizing beam splitter (6260) can direct the orthogonal states to the two line scan cameras (6270, 6280), which may be mounted on five-axis translation stages.

A polarization state that is transmitted straight through a polarizing beam splitter can be generally pure, e.g., approximately 99% of the power can be horizontally polarized. The polarization state that is reflected at 90° by a polarizing beam splitter can be less pure, with the horizontally polarized light mixing with vertically polarized light. Since such contamination may distort a proper polarization analysis, the horizontally polarized light can be filtered from the reflected polarization state using a cleanup polarizer. A Polarcor wire grid polarizer can be with an extinction ratio of 1:10,000 and a transmission performance of higher than about 90% over the full bandwidth. The polarizer may be positioned in front of the off-axis line scan camera (6270). The transmitted wavefront distortion of such polarizer may be specified as less than a quarter wavelength (at 632.8 nm). Spectra can be recorded simultaneously with the two line scan cameras (6270, 6280), and stored to the hard disk or any other storage device. An on-screen frame rate of approximately three frames per second can be maintained in real time. The polarization state in all arms of the interferometer can be optimized using the polarization controllers (6010, 6060, 6150, 6210).

It is further possible to utilize a prior art system described in B. Cense et al., “In vivo birefringence and thickness measurements of the human retinal nerve fiber layer using polarization-sensitive optical coherence tomography,” Journal of Biomedical Optics, 2004, Vol. 9(1), pp. 121-125 to simultaneously acquire OCT data and/or video images. As shown in FIG. 6, the PS-SD-OCT system according to the exemplary embodiment of the present invention can include a CCD camera (6170) that may be used to, e.g. position the scans around the optic nerve head. Such camera images do not have to be stored on the hard disk or any other storage device, or can be stored thereon if desired. Before and during the data acquisition described above, the information from the CCD camera (6170) and the real-time OCT structural intensity display can be used, e.g., to aim the scanning beam through the center of the pupil, and to position the scans around the optic nerve head. In addition, both imaging modalities can be used, e.g., to focus the beam onto the retina, guaranteeing data with the highest possible signal-to-noise ratio.

### Exemplary Calibration of Polarization-Sensitive Spectrometer

Generally, in the SD-OCT system, a reflectivity depth profile (A-line) can be obtained as the Fourier transform of a spectrum resulting from remapping from wavelength-space to k-space (k=2 π/λ). This remapping can depend on a knowledge of the wavelength that is incident on the different pixels of the line scan camera. An error Δλ of the assumed incident wavelength λ can be used to generate a deviation in a wave number provided by Δk=2 π Δk/λ². If the two line scan cameras have even slightly different errors, the relative deviation in the wave number can give rise to an artificial appearance of a birefringence. For an incident wavelength of λ=850 nm and with a relative alignment error of Δλ=1 nm between the cameras, a phase difference Δφ=8.70 radians over a depth of 1 mm can be obtained. The cumulative effect of these phase differences across the line scan cameras can lead to an overall phase difference that may not be distinguished from a phase retardation due to the sample birefringence. A removal of this artificial, or “ghost”, birefringence is likely beneficial to obtain a more accurate determination of sample polarization properties.

The relationship between the pixel position on the LSC and the corresponding wavelength λ can be obtained from the standard grating formula using simple geometry, and may be provided by the following equation:

$$λ = Δ x \{ sin ( {θ}_{i} ) + sin [ a sin ( \frac{{λ}_{c}}{Δ x} - sin ( {θ}_{i} ) ) - {tan}^{- 1} ( \frac{{x}_{CCD} - {x}_{0}}{F + dF ( 1 - D / F )} ) ] \}$$

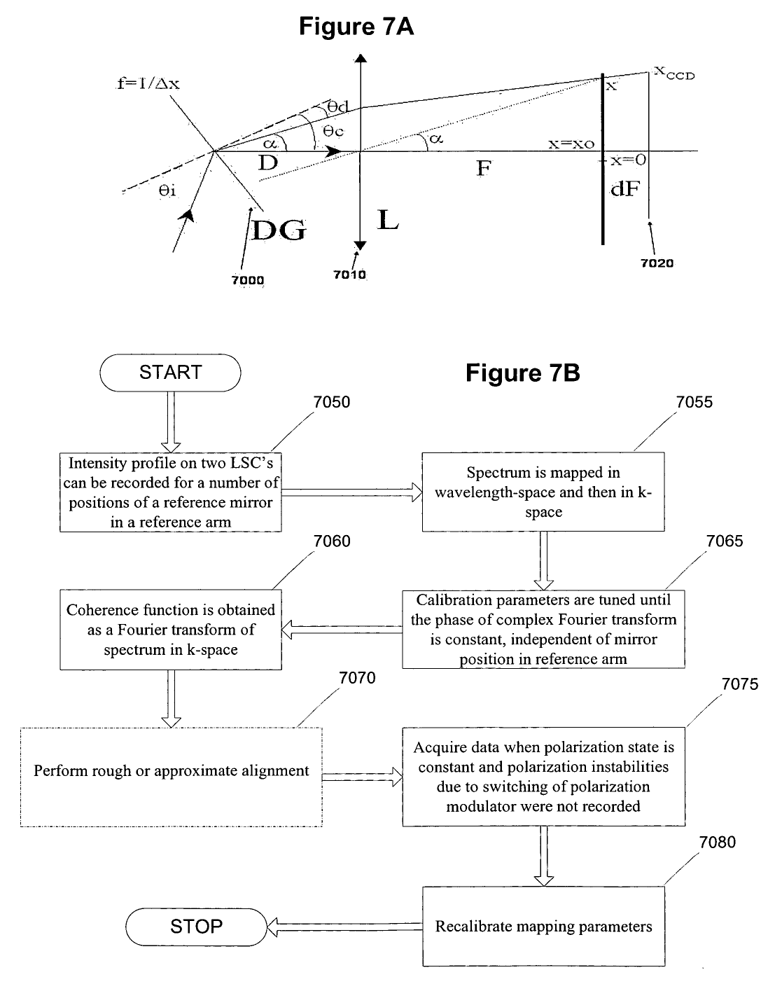

FIG. 7A illustrates an exemplary spectrometer configuration for one polarization channel in accordance with an exemplary embodiment of the present invention. A diffraction grating DG (7000) with the grating constant f=1/Δx can be provided. In addition, a focusing lens L (7010) with a focal length F may also be included in this exemplary configuration. As shown in FIG. 7A, θᵢ is the incident angle, θ_{d} is the diffraction angle. Further, λ_{c} denotes the central wavelength that is diffracted at an angle θ_{c} and propagates unbent through the focusing lens L being incident on a CCD (7020) on a pixel at a distance xₒ from the center of the CCD (7020) array (x=0). D denotes the distance between the grating (7000) and the focusing lens (7010), while dF represents a small displacement of the CCD (7020) from the focal plane of the lens (7010). This longitudinal displacement dF can be similar to or substantially equivalent to slightly tuning a focal length of the focusing lens (7010). Therefore, F can be considered a calibration parameter. The other calibration parameters are the incident angle θᵢ, the central wavelength λ_{c}, and a lateral shift x₀ of the CCD (7020).

In an exemplary two polarization channels configuration described above and shown in FIG. 7A, the incident angle θᵢ and the central wavelength λ_{c} can be substantially the same for the polarization channels because the beam splitter may be provided after the focusing lens, and the optical path can be common until the PBS. The parameters that may be related to the displacements of the two LSC's, F and xo, should preferably be different from one another. Thus, there may be a certain number of independent calibration parameters, e.g., θᵢ, λ_{c}, F₁, F₂, xₒ₁, and xₒ₂.

For a non-polarization-sensitive system according to another exemplary embodiment of the present invention, the exemplary procedure according to an exemplary embodiment of the present invention for determining the calibration parameters is provided below and shown in a flow diagram in FIG. 7B.

Initially, in step 7050, the intensity profile on the two LSC's is recorded for a number of positions of the reference mirror in the reference arm. In step 7055, the sample arm contains a mirror in a water-filled model eye to simulate a patient measurement. The spectrum may be mapped in wavelength-space and then in k-space (step 7060), and the coherence function can be obtained as the Fourier transform of the spectrum in k-space (step 7065). In step 7075, the calibration parameters can be tuned until the phase of the complex Fourier transform is constant, independent of the mirror position in the reference arm. This phase term can be used for a dispersion compensation for the patient measurement as described above.

Further, a rough alignment can be done in step 7070 and can be done performed prior to the data acquisition step 7075. The reference arm signal is maximized on both cameras. To align the two cameras with one another, a non-birefringent scattering sample (such as a stack of microscope cover slips or a uniformly scattering medium) can be imaged, and real-time polarization processing can be performed to, e.g., visually remove large amounts of the artificial birefringence. This can be performed by moving the location of one camera perpendicular to the beam until the observed birefringence, as measured with the exemplary embodiment of the system according to the present invention, becomes small or even negligible. This may insure the particular alignment of one camera with respect to the other, i.e., that the incident wavelength on corresponding pixels of the two line scans cameras can be approximately or roughly the same.

Second, a more careful recalibration of the mapping parameters can be performed in step 7080. This can be achieved, e.g., by optimizing various merit functions other than, or in addition to, the previous condition of constant phase of the complex Fourier transform independent of the mirror position in the reference arm. One such exemplary function can rely on the state of polarization of light (e.g., a Stokes vector) incident on the spectrometer. The stokes vector can be determined as described in J. F. de Boer et al., “Determination of the depth-resolved Stokes parameters of light backscattered from turbid media by use of polarization-sensitive optical coherence tomography,” Optics Letters, 1999, Vol. 24(5), pp. 300-302. The calibration parameters can be optimized such that the measured state of polarization is constant, independent of the mirror position in the reference arm. The set of calibration parameters and the phase factors for the two cameras may be subsequently used for correct mapping of the spectra in patient measurements and for dispersion compensation.

According to another exemplary embodiment of the present invention, the rough alignment described above with reference to step 7070 does not have to be performed. The appearance of the artificial birefringence can be eliminated by an appropriate calibration of the mapping parameters for the two cameras. However, without the rough alignment described above with reference to step 7070, the range over which parameters, such as x₀, vary, can be substantial. Thus, the rough alignment can make the optimization process easier and more beneficial.

### Exemplary and Experimental Measurement Procedures on Subjects

Certain experiments have been performed under a protocol that adhered to the tenets of the Declaration of Helsinki. For such experiments, one healthy volunteer and seven glaucoma patients were enrolled. Patients with various stages of open angle glaucoma (primary, pigmentary, and pseudoexfoliation forms) were obtained, and it was determined whether the patients were eligible for the study. After giving informed consent and determining that the patients were eligible to participate in the study, the eligible eyes of the glaucoma patients were dilated with phenylephrine hydrochloride 5.0% and tropicamide 0.8%. Measurements were performed on all enrolled subjects using the exemplary embodiments of the system, arrangement and method according to the present invention.

Healthy Subjects

For comparison, the healthy volunteer was previously imaged with both the prior polarization-sensitive time-domain system described in B. Cense et al., “In vivo depth-resolved birefringence measurements of the human retinal nerve fiber layer by polarization-sensitive optical coherence tomography,” Opt. Lett., 2002, Vol. 27(18), pp. 1610-1612, B. Cense et al., “In vivo birefringence and thickness measurements of the human retinal nerve fiber layer using polarization-sensitive optical coherence tomography,” Journal of Biomedical Optics, 2004, Vol. 9(1), pp. 121-125, and B. Cense et al., “Thickness and birefringence of healthy retinal nerve fiber layer tissue measured with polarization-sensitive optical coherence tomography,” Investigative Ophthalmology & Visual Science, 2004, Vol. 45(8), pp. 2606-2612, as well as the spectral-domain system described in N. Nassif et al., “In vivo human retinal imaging by ultrahigh-speed spectral domain optical coherence tomography,” Optics Letters, 2004, Vol. 29(5), pp. 480-482, N. A. Nassif et al., “In vivo high-resolution video-rate spectral-domain optical coherence tomography of the human retina and optic nerve,” Optics Express, 2004, Vol. 12(3), pp. 367-376, and B. Cense et al., “Ultrahigh-resolution high-speed retinal imaging using spectral-domain optical coherence tomography,” Optics Express, 2004.

For this experiment, the power of the light incident on the volunteer's undialated right eye was equal to 470 μW. Two different types of scans were performed around the optic nerve head. One data set was made with concentric circular scans (12 circular scans of 1000 A-lines equidistantly spaced between 1.5 and 2.6 mm radius), the other data set was made with 250 linear scans of 500 A-lines covering an area of 6.4×6.4 mm. Data was acquired at integration times of either 33 μs or 132 μs per A-line. For the last set, the speed at which the exemplary system was operating has been reduced by a factor of 4, thus improving the sensitivity by a factor of 4. This setting was still almost 45 times faster than the time-domain measurement, therefore reducing the total measurement time for 12 circular scans from 72 seconds to 1.6 s. The eye that was under investigation was stabilized with a fixation spot.

Glaucoma Patients

The power incident on the eye was less than 500 μW for the glaucoma patients. In cases where the patient could only see with one eye, the eye that lacked vision was imaged. The eyes that were imaged were stabilized with the internal fixation light of the slit lamp system. An external fixation light was used for the contralateral eye of the patients who could not see this light. Circular scans of 1000 A-lines with integration times of 33 and 132 μs were performed. In addition, some eyes of these patients were imaged with an integration time of 330 μs. Further, linear scans (200 scans of 1000 A-lines, 6.4×6.4 mm) were performed at 132 μs per A-line.

Exemplary Data Analysis

The polarimetric analysis consisted of several procedures. In the first exemplary procedure, the spectrometer was calibrated as described above. The calibration parameters were used for mapping the measured spectra to wavelength-space, and then to k-space. In addition, the phase curve determined for each camera was used to compensate for chromatic dispersion in the eye and the interferometer, as described in R. Chan et al., “Anisotropic edge-preserving smoothing in carotid B-mode ultrasound for improved segmentation and intima-media thickness measurement,” Computers in Cardiology, Cambridge, Mass., IEEE, 2000. After Fourier transforming the data to z-space, the depth-resolved Stokes parameters were determined as described M. C. Pierce et al., “Simultaneous intensity, birefringence, and flow measurements with high-speed fiber-based optical coherence tomography,” Optics Letters, 2002., Vol. 27(17), pp. 1534-1536. The first depth-resolved Stokes parameter corresponds to the structural intensity, e.g., a depth resolved reflectivity. The upper and lower boundaries of the retinal nerve fiber layer were determined from this data as described in R. Chan et al., “Anisotropic edge-preserving smoothing in carotid B-mode ultrasound for improved segmentation and intima-media thickness measurement,” Computers in Cardiology, Cambridge, Mass., IEEE, 2000. In the polarization analysis, the normalized surface Stokes vectors were compared with the normalized Stokes vectors at a certain depth to determine the depth-resolved phase retardation, as described in C. E. Saxer et al., “High-speed fiber-based polarization-sensitive optical coherence tomography of in vivo human skin,” Optics Letters, 2000, Vol. 25(18), pp. 1355-1357, B. Cense et al., “In vivo depth-resolved birefringence measurements of the human retinal nerve fiber layer by polarization-sensitive optical coherence tomography,” Opt. Lett., 2002, Vol. 27(18), pp. 1610-1612, B. Cense et al., “In vivo birefringence and thickness measurements of the human retinal nerve fiber layer using polarization-sensitive optical coherence tomography,” Journal of Biomedical Optics, 2004, Vol. 9(1), pp. 121-125, and B. Cense et al., “Thickness and birefringence of healthy retinal nerve fiber layer tissue measured with polarization-sensitive optical coherence tomography,” Investigative Ophthalmology & Visual Science, 2004, Vol. 45(8), pp. 2606-2612.

For the data obtained from a healthy volunteer, the surface Stokes vector was selected to be 10 μm below the automatically-detected surface, and for the glaucoma patient, a value of 3 μm has been selected to preserve as many points as possible for accurate data extraction. Moving-average filters were used to reduce the influence of speckle noise. In the horizontal direction over 20 A-lines were averaged, while in the vertical direction over 3 points were averaged which corresponds to 10 μm. The thickness and birefringence of the retinal nerve fiber layer tissue was measured as a function of sector and radius. Each circular scan was divided in 50 sectors of 7.2°. The 50 sectors almost matched the 48 sectors that were used for the time-domain data.

Data sets that were acquired with linear scans were processed into a surface image, substantially equivalent to those made with either a fundus camera, a scanning laser ophthalmoscope or with a scanning laser polarimeter. This was performed by summing intensity values per A-line to one value corresponding to an integrated reflectivity along each depth profile. Fir example, a three-dimensional volume data set can be projected to a two-dimensional image, which appears as a fundus image.

### Exemplary Experimental Results

Results Obtained From a Healthy Subject

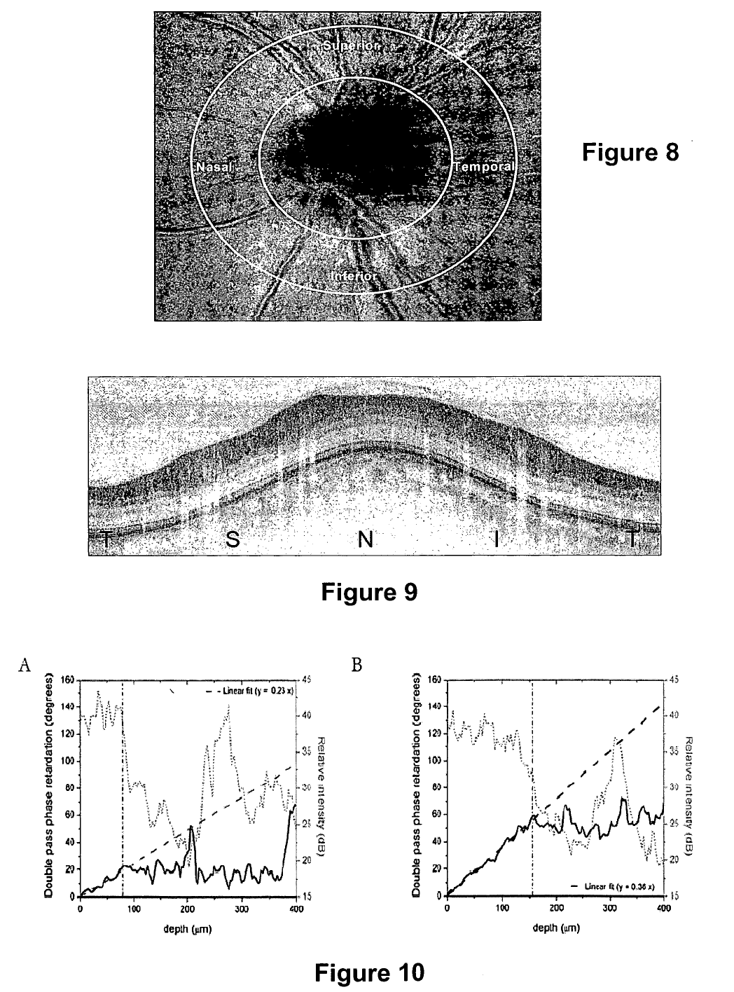

A set of linear scans (6.4×6.4 mm, 500×250 data points, acquired at 7.5 kHz), processed in a fundus-like image using the exemplary embodiment of the present invention is illustrated in FIG. 8. In particular, FIG. 8 shows an obtained exemplary pseudo fundus image of the optic nerve head, reconstructed from a three-dimensional volume set. White circles indicate the approximate positions of the smallest and largest diameter circular scans. Large blood vessels can be seen branching out from the optic nerve in the superior and inferior areas.

For example, circular scans made at 30 kHz and 7.5 kHz were analyzed and compared with each other. The 7.5 kHz data set demonstrated a higher signal-to-noise ratio (˜41 dB vs. ˜36 dB), and did not contain noticeable motion artifacts. FIG. 9 shows a structural intensity image of a circular scan around the optic nerve head of a healthy volunteer taken at A-line rate of 7.5 kHz with a circular scan of the undilated right eye of a 40-year-old healthy volunteer. As shown in FIG. 9, positions in the eye are labeled: temporal (T); superior (S); nasal (N); inferior (I). The image measures 0.96 mm deep by 12.6 mm wide and is expanded in vertical direction by a factor of four for clarity. The image was not realigned, and shows the true topography of the tissue around the optic nerve head. The dynamic range of the image above the noise floor was 38.5 dB. The horizontal lines below the top of the image were caused by electrical noise in the off-axis line scan camera.

The dynamic range of the image is 38.5 dB (in the same data set, images with a dynamic range up to 44 dB were found). Strong reflections are represented by black pixels in FIG. 9. The image was expanded in the vertical direction for clarity. As described in B. Cense et al., “Thickness and birefringence of healthy retinal nerve fiber layer tissue measured with polarization-sensitive optical coherence tomography,” Investigative Ophthalmology & Visual Science, 2004, Vol. 45(8), pp. 2606-2612, the superior (S) and inferior (I) areas contain RNFL tissue that is relatively thick.

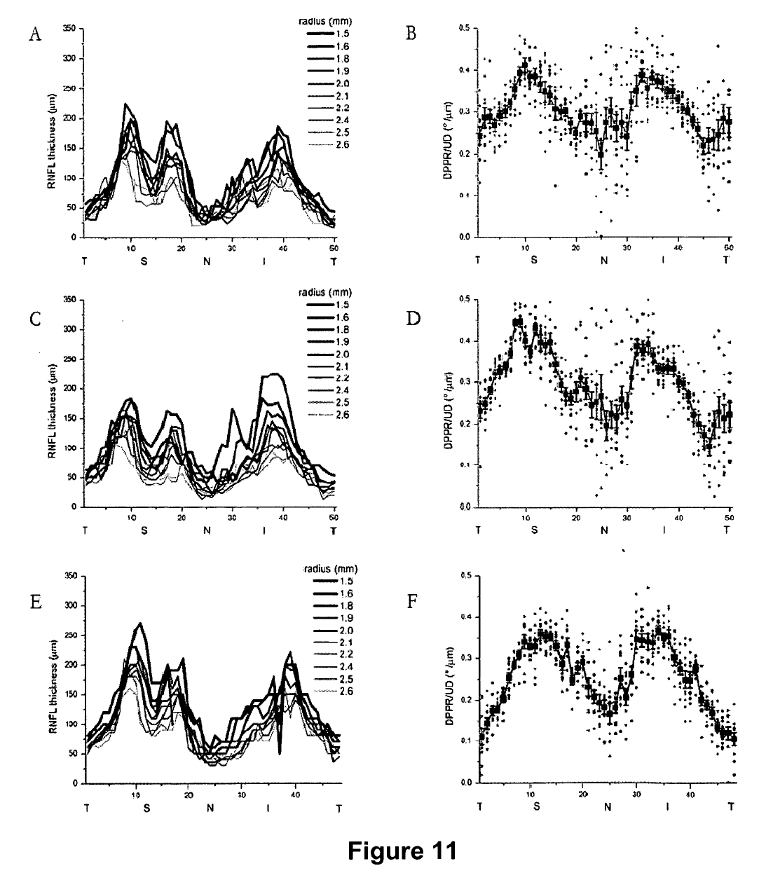

Both data sets were analyzed to compare the thickness and double-pass phase retardation per unit depth (DPPR/UD) as a function of sector and radius. The data set acquired at 30 kHz was compared with the one taken at 7.5 kHz, as well as with the data set that was previously acquired with the time-domain system at 256 Hz. FIGS. 11A-11F show the graphs of these exemplary measurements, e.g., RNFL thickness and DPPR/UD measurements at different integration times. For example, FIGS. 11A and 11B illustrate graphs of data obtained at 7.5 kHz, and FIGS. 11C and 11d show data taken at 30 kHz. FIGS. 11E and 11F are shown for comparison purposes, as being taken at 256 Hz with the time-domain OCT system. The thickness graphs shown in FIGS. 11A, 11C and 11E have been developed similarly, with a double-hump pattern and higher values superiorly (S) and inferiorly (I). In the superior area, a smaller double-hump pattern can be seen in FIG. 11C. The DPPR/UD graphs develop similarly with high values superior and inferior. The spread of measurement points around the mean values (e.g., connected with a line) is likely higher for the spectral-domain data as shown in FIGS. 11B and 11D than for the time-domain data shown in FIG. 11F.

The spectral-domain OCT measurements averaged over one sector are discussed below, starting with a measurement in the temporal section, taken from the data shown in FIGS. 10A and 10B. In particular, FIG. 10A illustrates a first exemplary graph illustrating a thickness and double-pass phase retardation (DPPR) of sectors temporal to ONH generated using the arrangement, system and/or method in accordance with an exemplary embodiment of the present invention. FIG. 10B shows a first exemplary graph of the thickness and DPPR of sectors superior to the ONH generated using the arrangement, system and/or method in accordance with an exemplary embodiment of the present invention. The thickness (e.g., shown as a dotted line) and DPPR (e.g., shown as a solid line) graphs of sectors temporal (A) and superior (B) to the ONH, were acquired with an A-line rate of 7.5 kHz. The data was averaged over a sector of 20 A-lines or 7.5°. DPPR data belonging to the RNFL may be fit with a least-squares linear fit. The slope in the equation represents the DPPR/UD. The vertical line indicates the estimated boundary of the RNFL, as determined from the intensity and DPPR data. The increase in DPPR at depths over 150 μm is caused by a low signal between the RNFL and the RPE.

For example, in the temporal area, the RNFL is thin and a relatively low DPPR/UD value can be obtained. The superior sector contains thicker RNFL tissue with a higher birefringence. Nasal plots demonstrate thin RNFL and low birefringence, while inferior plots show thick RNFL with high DPPR/UD values. Thickness values were plotted as a function of radius and sector, and data points taken at one radius were connected with a line. The thickness of the line indicates the radius of the scan, with thicker lines of scans closer to the optic nerve head. DPPR/UD values were also plotted as a function of radius and sector, with data points at a certain radius bearing the same symbol. The mean DPPR/UD value per sector was determined and a line connected mean values per sector. The standard error (SE) of the mean was determined and is represented in the graphs by error bars.

Comparing the thickness graphs of FIGS. 11A-11F, a similar trend can be observed, with higher values superiorly and inferiorly. The higher thickness values in these areas can be explained by the presence of arcuate nerve fiber bundles, which branch off towards the fovea. The differences in the thickness measurements can be attributed to subjective interpretation of the data by the operator. An automatic image analysis procedure in accordance with one exemplary embodiment of the present invention may improve the objectivity and the analysis. The DPPR/UD graphs show similar trends as well, with higher values superiorly and inferiorly. The SD-OCT data results obtained at 7.5 kHz can better match with the TD-OCT data results. The temporal values can increase in both SD-OCT data sets, while these results are low in the TD-OCT setup. The general trend of the higher inferior and superior values can be seen in all graphs of FIGS. 11A-11F. The maximum mean DPPR/UD value measured in this subject with PS-SD-OCT was approximately 0.45°/μm, while the minimum mean value equals to approximately 0.2°/μm. Such values may be approximately equivalent to the birefringence of 5.4×10⁻⁴ and 2.4×10⁻⁴, respectively, measured at 840 nm.

Discussion of Exemplary Results Obtained From the Healthy Subject

Comparing the time-domain DPPR/UD plot shown in FIG. 11F with the spectral-domain plots shown in FIGS. 11B and 11D, the spectral-domain data points are shown to be scattered over a larger range. This may be partly due to imperfect use by an operator of the exemplary embodiments of the system, arrangement and method of the present invention when used for the spectral-domain data, by the use of an automatic slope-fitting procedure and by averaging over a relatively low number of A-lines. For noisy time-domain measurements, the average DPPR value below the RNFL was used to calculate the DPPR/UD. The average DPPR value can be divided by the thickness of the RNFL to calculate the DPPR/UD. For the spectral-domain values, the procedure can fit a line through the DPPR data points of the RNFL, independent of noise present on the data. For the thick parts of the RNFL, with many data points to fit, this exemplary procedure likely yields reliable results.

Results of a glaucoma subject

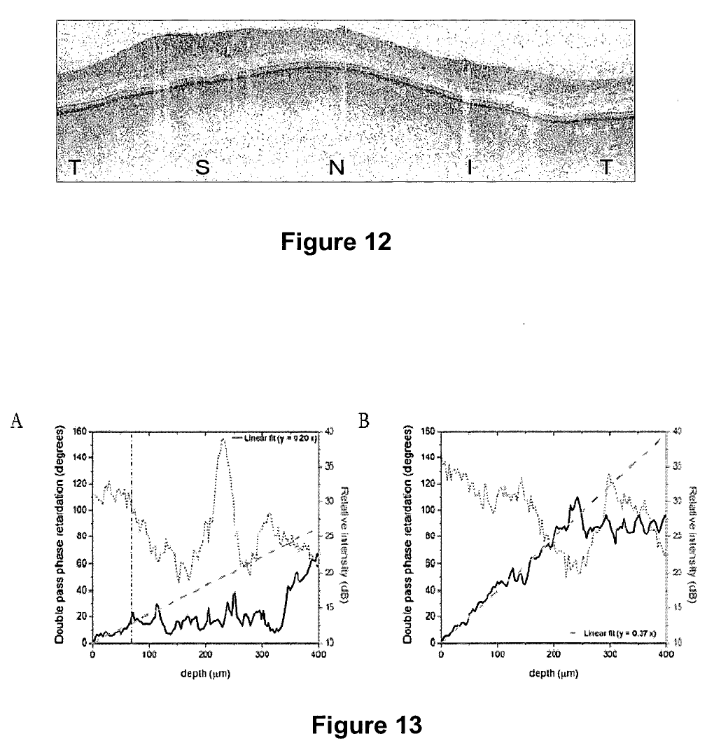

The glaucoma patients were imaged with the exemplary PS-SD-OCT system, arrangement and method. A particular data set had a signal-to-noise ratio that was beneficial to be analyzed. This data set was obtained from the left eye of an 81-year old white female. She had undergone cataract surgery 6 years earlier, which possibly lead to the relatively high image quality. Her best-corrected visual acuity was 20/20, and the internal fixation spot was used to stabilize the eye. The visual field test results showed a superior visual field defect, which should result in a thinner nerve fiber layer in the inferior area (i.e., the vision of the eye may be inverted). The reported field defect was relatively small. FIG. 12 shows an exemplary tructural intensity image taken from a circular scan around the optic nerve head of this glaucoma patient. The image shows a relatively thin inferior nerve fiber layer (I), caused by glaucoma. All other areas appear to be unaffected. The positions in the eye are labeled as follows: temporal (T); superior (S); nasal (N); inferior (I). This image measures 0.96 mm deep by 12.6 mm wide and is expanded in the vertical direction by a factor of four for clarity. The dynamic range of the image above the noise floor was 37.4 dB, with A-lines acquired at 7.5 kHz. The image was taken at a radius of 2 mm and an A-line acquisition rate of 7.5 kHz.

Compared to the scans made in the healthy subjects (e.g., the image shown in FIG. 9), the contrast between the RNFL and ganglion cell layer, which borders the RNFL, is not as strong. The inferior (I) RNFL tissue of this patient is thinner than the equivalent inferior tissue of the healthy subject.

FIG. 13A shows a second exemplary graph of the thickness and DPPR of sectors temporal to the ONH generated using the arrangement, system and/or method in accordance with an exemplary embodiment of the present invention, and FIG. 13B illustrates a second exemplary graph of the thickness and DPPR of the sectors superior to the ONH generated using the arrangement, system and/or method in accordance with an exemplary embodiment of the present invention. The data provided in FIGS. 13A and 13B was obtained from a glaucoma patient. DPPR data in each graph belonging to the RNFL is fit with a least-squares linear fit. The slope in the equation represents the DPPR/UD. The vertical line indicates the estimated boundary of the RNFL, as determined from the intensity and DPPR data.

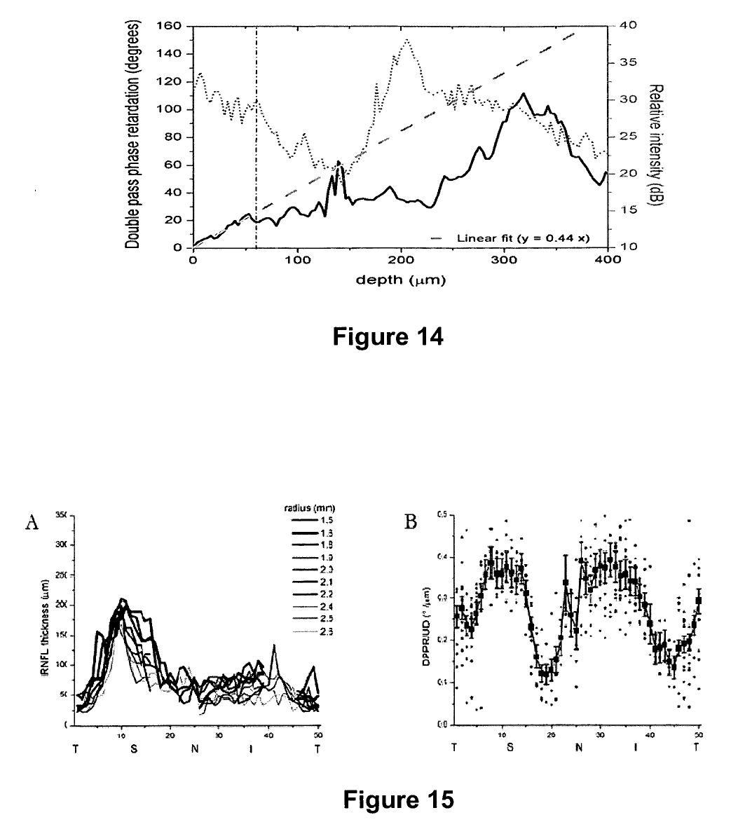

In the structural intensity image shown in FIG. 12, a field defect was observed in the inferior area (labeled “I”). FIG. 14 shows graphs of the DPPR results (solid line) and thickness (dotted line) from a sector within this field defect in the inferior area of the glaucoma patient. Although the RNFL shows to be relatively thin, the DPPR/UD remains high.

After analyzing all sectors at all radii, thickness and DPPR/UD plots were combined in two graphs. For example, the thickness graph shown in FIG. 15A indicates that the thickness measured in the superior area decreases as a function of radius. This decrease was also seen in the healthy subjects, as described in B. Cense et al., “Thickness and birefringence of healthy retinal nerve fiber layer tissue measured with polarization-sensitive optical coherence tomography,” Investigative Ophthalmology & Visual Science, 2004, Vol. 45(8), pp. 2606-2612, and B. Cense et al., “Thickness and birefringence of retinal nerve fiber layer of healthy and glaucomatous subjects measured with polarization sensitive optical coherence tomography,” Ophthalmic Technologies XIV, Proceedings of SPIE Vol. 5314, 2004, pp. 179-187. In the inferior area, this decrease as a function of radius may be less clear, since curves from different radii overlap. When compared to the thickness graph of the healthy subjects, the inferior area of the glaucoma subject is thinner. In particular, the ratio between the superior and inferior area is significantly larger in this glaucoma patient than in the healthy subject. The thinner inferior area is in agreement with the visual field defect as measured with the visual field test.

The DPPR/UD graph shown in FIG. 15B indicates high superior (S) values. High values can also be obtained between the nasal (N) and inferior (I) areas, while low values occur in the nasal and temporal area. Between the temporal and inferior area, a depression is evident. The general trend is similar to the trends observed in the healthy subjects, when used with both the spectral-domain and time-domain OCT systems and procedures.

Based on the analysis of the results from the healthy subjects, another averaging procedure has been developed in accordance with a further exemplary embodiment of the present invention to reduce the possible effects of the slightly noisier DPPR graphs. For example, according to this procedure, the data was analyzed again, and an averaging filter has been implemented to average the Stokes parameters of 40 A-lines. Data was consequently mapped over fewer data points in the scan, decreasing the number of sectors by a factor of 2.

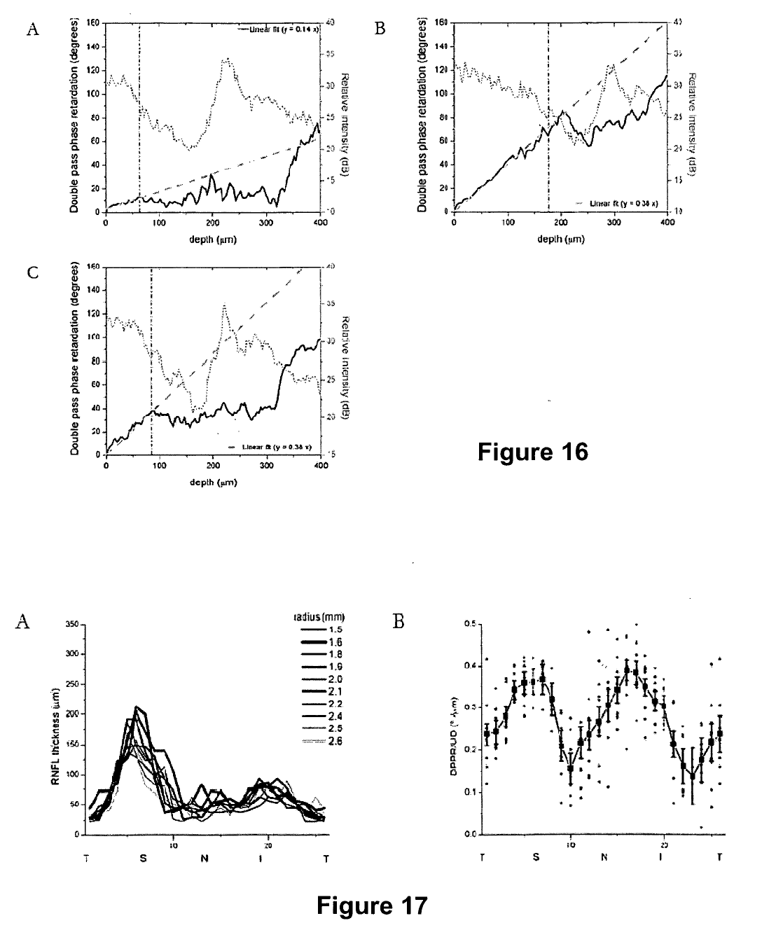

FIG. 16A shows an exemplary graph providing the thickness (dotted line) and DPPR (solid line) plots of an area nasal to the ONH of the glaucoma patient, FIG. 16B illustrates an exemplary graph providing the thickness and DPPR plots of an area superior to the ONH of the glaucoma patient, and FIG. 16C shows an exemplary graph providing the thickness and DPPR plots of an area inferior to the ONH of the glaucoma patient. These graphs demonstrate DPPR/UD values that are similar to those displayed in the graphs of FIGS. 15A and 15B. For these graphs, the Stokes parameters of 40 A-lines were averaged to reduce the influence of speckle noise. Comparing these graphs with the sector graphs of the same patient that were averaged over fewer A-lines (shown in FIGS. 13A and 13B), these curves are less noisy. The results of all sectors and radii are shown in FIGS. 17A and 17B. In particular, FIG. 17A shows an exemplary graph providing the RNFL thickness from the nerve fiber layer tissue of the glaucoma patient, and FIG. 17B illustrates an exemplary graph providing the DPPR/UD values from the nerve fiber layer tissue of the glaucoma patient. For these graphs, the Stokes parameters from 40 A-lines were averaged. The trends that could be seen in glaucoma data averaged over 20 A-lines remain the same: high DPPR/UD values superiorly and inferiorly, with the thickest tissue located in the superior area. While the averaging procedure reduces the spread in data points, the overall trend remains very similar.

The maximum mean DPPR/UD value measured in this patient with the PS-SD-OCT systems and procedures was approximately 0.4°/μm, while the minimum mean value may be approximately 0.15°/μm. These values are approximately equivalent to a birefringence of 4.8×10⁻⁴ and 1.8×10⁻⁴, respectively, measured at 840 nm.

Discussion of Results of the Glaucoma Subjects

According to the exemplary embodiments of the present invention, it is believed that glaucoma causes a decrease of the RNFL birefringence, since less birefringent amorphous glial cells would replace the well aligned and birefringent nerve fibers. Although the inferior area of the glaucoma patient may be relatively thin as a result of glaucoma, most of the DPPR/UD values in this area appeared normal. There was a slight depression in the region between the inferior and temporal area, which can be observed in some healthy subjects as well, but between the nasal and inferior areas, normal inferior values occur. The peak value of approximately 0.4°/μm is very similar to the DPPR/UD value in the superior area, and those of the inferior and superior area of the healthy subjects.

Most of the RNFL in the inferior area is only slightly thicker than 75 μm. For a time-domain measurement at the same signal-to-noise ratio, the DPPR/UD measurements are generally reliable. However, these measurements were obtained at a lower signal-to-noise ratio than measurements obtained from the healthy subject (shown in FIGS. 11B and 11D). Indeed, the signal-to-noise ratio of the glaucoma data was on average approximately 3 dB lower than the data from the healthy subject. Such exemplary results were obtained from one glaucoma patient with one type of glaucoma, and can be useful for all glaucoma patients.

Further, a higher signal-to-noise ratio (SNR) can be achieved in several ways in accordance with the exemplary embodiments of the present invention. As an initial matter, SNR can be improved by increasing the source arm power. The ANSI standards provide for a use of a higher power than 600 μW for the scanning beams. At an acquisition speed of 7.5 kHz, a scan length of 9.4 mm (scan with the shortest radius) and a scan time of 132 ms per scan, the power can be increased by a factor of 15 to approximately 9 mW. Further, it is possible to reduce the scan rate, without increasing the power. For example, reliable DPPR/UD results can be obtained by slowing down the scan rate to about 3 kHz. A longer acquisition time may become problematic for the glaucoma patients, since motion artifacts are more likely to occur. A retina tracker can avoid such artifacts, and also automatically rescan areas that were missed because of blinks, as described in R. D. Ferguson et al., “Tracking optical coherence tomography,” Optics Letters, 2004, Vol. 29(18), pp. 2139-2141. Since spectral-domain measurements in the healthy subject match well with those obtained in the time-domain measurements, another option can be to perform the exemplary procedures according to the present invention on young subjects with glaucoma.

### Exemplary Experimental Conclusions

The birefringence of a healthy RNFL tissue, measured in one healthy subject with spectral-domain polarization-sensitive OCT systems, arrangements and methods according to exemplary embodiments of the present invention, can be constant as a function of scan radius, and may vary as a function of position around the ONH, with higher values occurring superior and inferior to the ONH. The measured mean DPPR/UDs around the ONH in one healthy subject varied between 0.20 and 0.45°/μm. These values may be equivalent to birefringence of 2.4×10⁻⁴ and 5.4×10⁻⁴, measured at a wavelength of 840 nm.

Measurements in a glaucoma subject with a small visual field defect demonstrate nerve fiber layer thinning in inferior sectors due to glaucoma. The polarization-sensitive measurements according to the exemplary embodiments of the present invention likely indicate that a portion of the nerve fiber layer tissue in these sectors is as birefringent as the healthy tissue.

### EXEMPLARY USES AND APPLICATIONS

Certain exemplary systems, arrangements, products, processes, services, procedures or research tools which can be used together with or incorporate the exemplary embodiments of the system, arrangement and method according to the present invention can include, but not limited to:
i. PS-SD-OCT system for early detection of glaucoma, as described in B. Cense et al., “In vivo depth-resolved birefringence measurements of the human retinal nerve fiber layer by polarization-sensitive optical coherence tomography,” Opt. Lett., 2002, Vol. 27(18), pp. 1610-1612, B. Cense et al., “In vivo birefringence and thickness measurements of the human retinal nerve fiber layer using polarization-sensitive optical coherence tomography,” Journal of Biomedical Optics, 2004, Vol. 9(1), pp. 121-125, and B. Cense et al., “Thickness and birefringence of healthy retinal nerve fiber layer tissue measured with polarization-sensitive optical coherence tomography,” Investigative Ophthalmology & Visual Science, 2004, Vol. 45(8), pp. 2606-2612,
ii. PS-SD-OCT system for obtaining corneal birefringence measurements,
iii. PS-SD-OCT system for providing a burn-depth analysis as described in B. H.

Park et al, “In vivo burn depth determination by high-speed fiber-based polarization sensitive optical coherence tomography,” Journal of Biomedical Optics, 2001, Vol. 6(4), pp. 474-9, and to perform a skin cancer detection by measuring the collagen content of the skin as described in M. C. Pierce et al., “Birefringence measurements in human skin using polarization-sensitive optical coherence tomography,” Journal of Biomedical Optics, 2004, Vol. 9(2), pp. 287-291, and M. C. Pierce et al., “Advances in Optical Coherence Tomography Imaging for Dermatology,” J Invest Dermatology, 2004, Vol. 123(3), pp. 458-463,
iv. PS-SD-OCT system for performing an optical diagnostic of the cardiovascular system disease by measuring the collagen content of coronary arteries,
v. PS-SD-OCT system for performing early diagnostic of tumors and cancerous tissue, and/or
vi. PS-SD-OCT system for performing measurements for quality control of scattering materials such as plastics, glasses and tissue.

The foregoing merely illustrates the principles of the invention. Various modifications and alterations to the described embodiments will be apparent to those skilled in the art in view of the teachings herein. Indeed, the arrangements, systems and methods according to the exemplary embodiments of the present invention can be used with any OCT system, OFDI system, SD-OCT system or other imaging systems, and for example with those described in International Patent Application PCT/US2004/029148, filed Sep. 8, 2004, U.S. patent application Ser. No. 11/266,779, filed Nov. 2, 2005, and U.S. patent application Ser. No. 10/501,276, filed Jul. 9, 2004, the disclosures of which are incorporated by reference herein in their entireties. It will thus be appreciated that those skilled in the art will be able to devise numerous systems, arrangements and methods which, although not explicitly shown or described herein, embody the principles of the invention and are thus within the spirit and scope of the present invention. In addition, to the extent that the prior art knowledge has not been explicitly incorporated by reference herein above, it is explicitly being incorporated herein in its entirety. All publications referenced herein above are incorporated herein by reference in their entireties.

What is claimed is:

1. An apparatus for separating an electromagnetic radiation, comprising:
a first arrangement configured to separate the electromagnetic radiation into at least one first portion and at least one second portion according to at least one polarization and at least one wave-length of the electromagnetic radiation; and
a second arrangement configured to simultaneously detect the separated first and second portions.

2. The apparatus according to claim 1, wherein the second arrangement comprises a detection arrangement which includes a single row of detection elements.

3. The apparatus according to claim 1, wherein the second arrangement comprises a first detection arrangement and a second detection arrangement, wherein each of the first and second detection arrangements includes a single row of detection elements.

4. The apparatus according to claim 1, wherein the first arrangement includes a first element which is configured to separate the electromagnetic radiation into the first and second portions based on the at least one polarization, and a second element which is configured to separate the electromagnetic radiation into the first and second portions based on the at least one wave-length.

5. The apparatus according to claim 4, wherein the first element follows the second element in an optical path of the electromagnetic radiation.

6. The apparatus according to claim 5, wherein the first arrangement further includes a third light directing element which is provided in the optical path in a proximity of the first and second elements.

7. The apparatus according to claim 6, wherein the third element is provided between the first and second elements.

8. The apparatus according to claim 6, wherein the third element follows the first and second elements in the optical path.

9. The apparatus according to claim 5, wherein the first arrangement further includes third and fourth light directing elements which are provided in the optical path following the first and second elements.

10. The apparatus according to claim 9, wherein each of the third and fourth elements directs at least one of the respective separated portions toward the second element.

11. The apparatus according to claim 4, wherein the second element follows the first element in an optical path of the electro-magnetic radiation.

12. An apparatus for obtaining information for a sample using an electromagnetic radiation, comprising:
a first arrangement configured to generate the electromagnetic radiation;
a second interferometric arrangement configured to receive and combine a first radiation from the sample and a second radiation from a reference into a further radiation, the first and second radiations being associated with the electromagnetic radiation; and
a third arrangement configured to separate the further radiation into at least one first portion and at least one second portion according to at least one polarization and at least one wave-length of the electromagnetic radiation; and
a fourth arrangement configured to simultaneously detect the separated first and second portions, and obtain the information as a function of the separated first and second portions.

13. The apparatus according to claim 12, wherein the first arrangement includes a further arrangement configured to control a polarization of the generated electromagnetic radiation.

14. The apparatus according to claim 12, wherein the fourth arrangement comprises a detection arrangement which includes a single row of detection elements.

15. The apparatus according to claim 12, wherein the third arrangement includes a first element which is configured to separate the electromagnetic radiation into the first and second portions based on the at least one polarization, and a second element which is configured to separate the electromagnetic radiation into the first and second portions based on the at least one wave-length.

16. The apparatus according to claim 15, wherein the first element follows the second element in an optical path of the electromagnetic radiation.

17. The apparatus according to claim 16, wherein the third arrangement further includes a third light directing element which is provided in the optical path at least one of (i) in a proximity of the first and second elements, or (ii) following the first and second elements.

18. The apparatus according to claim 17, wherein the third element at least one of (i) is provided between the first and second elements, or (ii) follows the first and second elements in the optical path.

19. The apparatus according to claim 17, wherein each of the third and fourth elements directs at least one of the respective separated portions toward the second element.

20. The apparatus according to claim 15, wherein the second element follows the first element in an optical path of the electromagnetic radiation.

21. A method for separating an electromagnetic radiation, comprising:
separating the electromagnetic radiation into at least one first portion and at least one second portion according to at least one polarization and at least one wave-length of the electro-magnetic radiation; and
simultaneously detecting the separated first and second portions.

22. A method for obtaining information for a sample using an electromagnetic radiation, comprising:
receiving and combining a first radiation from the sample and a second radiation from a reference into a further radiation, the first and second radiations being associated with the electromagnetic radiation;
simultaneously detecting first and second portions of the further radiation that were separated from the further radiation according to at least one polarization and at least one wave-length of the electromagnetic radiation; and
obtaining the information as a function of the separated first and second portions.
